(19) United States
(12) Patent Application Publication
(10) Pub. No.: US 2021/0379247 A1
(43) Pub. Date: Dec. 9, 2021

(54) TISSUE MECHANICAL FRAGMENTATION DEVICE INTENDED FOR THE PREPARATION OF A COMPOSITION OF ISOLATED CELLS, AND CORRESPONDING METHOD

(71) Applicant: STEMCIS, SAINTE-CLOTILDE (FR)

(72) Inventors: Régis ROCHE, Pouilley-les-Vignes (FR); Franck FESTY, LA MONTAGNE (FR)

(21) Appl. No.: 16/629,792

(22) PCT Filed: Jul. 12, 2018
(86) PCT No.: PCT/FR2018/051763

§ 371 Date: Jan. 9, 2020

(30) Foreign Application Priority Data
Jul. 12, 2017 (FR) 1756643

(51) Int. Cl. A61L 27/36; C12M 1/33; C12M 1/00; A61L 27/38; C12N 5/077

(52) U.S. Cl. CPC A61L 27/3691; C12M 45/02; C12N 2509/10; A61L 27/3804; C12N 5/0653; C12M 47/04

(57) ABSTRACT

A device for mechanical fragmentation of tissues facilitates preparation of the composition of isolated cells from a tissue sample, the preparation including mechanical fragmentation of the tissue, the device including a motorized support and a container, the motorized support including a motor having a rotor secured to a coupling member, the container internally including a rotary shaft with radial arms sweeping the inside of the container as the shaft rotates, the motorized support and the container including a complementary securing unit that allow the container to be immobilized on the motorized support, the coupling member and the rotary shaft being configured in such a way as to allow axial rotation of the rotary shaft when the rotor of the motor is turning. The radial arms are of two types: rigid vane-type arms, and flexible filamentary-type arms, notably made of nylon or of metal. A corresponding method is also disclosed.

### TECHNICAL FIELD TO WHICH THE INVENTION RELATES

The present invention generally relates to the field of devices for preparing cell compositions. It more particularly relates to a device for the mechanical fragmentation of tissues intended for the preparation of a composition of isolated cells, as well as a method using the device.

### TECHNOLOGICAL BACK-GROUND

It is known to proceed, for various purposes, to injections of living cells into certain parts of the human or animal body. These cells have properties that allow an action on injured tissues or organs, by regeneration or symptomatic treatment, for example, of pain.

Methods for obtaining such cells have hence been developed, in particular for adipocytes. Generally, these methods for obtaining such adipocyte cells have a common step based on the enzymatic digestion of the extracellular matrix of the tissue by a cocktail of proteases (collagenases) in order to release the cells from the tissue.

Among the known methods, document WO 2007/034115 A1 may be mentioned, which describes both a method and tools for purifying certain cells contained in adipose tissue.

The methods described in documents WO 2015/035221 A1; US 2005/139704 A1 and WO 95/09051 A1 are also known.

Such cell release by this enzymatic method is efficient but this type of enzymatic method is subjected to regulatory constraints and is very expensive.

To avoid these constraints, purely mechanical methods may be used, which make it possible to purify the adipocyte cells without the use of enzyme. Most of the known mechanical methods use a mechanical action in order to release the cells from the matrix structure of the tissue. The devices allowing these operations are considered as Class II Medical Devices.

In the field of preparation of cells from tissue samples, it may be mentioned, in 2014, a StromaCell® system from MicroAire, which is described as a semi-automated method for mechanically isolating cells from the adipose tissue with 140 000 cells per millilitre of lipoaspirate. A publication from M. Rapisio's team, of the same year, describes a vibration isolation in a pot during 6 min, followed by a centrifugation to collect the cells with a yield of 125 000 cells per millilitre of lipoaspirate, hence 5% of progenitor cells (Aronowitz et al., SpringerPlus, 2015).

Thereafter, devices claiming the isolation of cells derived from the adipose tissue have arrived in mass. More than ten systems have been listed by Oberbauer in 2105 (Oberbauer et al., Cell Regeneration, 2015), the most known being PureGraft (Cytori), Fastem (Corios Soc Coop), Revolve/GID 700 (LifeCell Corporation/GID Group Inc), Lipogems (Lipogems International S.p.A.), StromaCell (MicroAire Surgical Instruments LLC) and MyStem (MyStem LLC). In 2016, Cicione's team has described cell isolation with the MyStem EVO technique (Cicione et al., PRS, 2016), and Domenis's team has made a comparative study of the Fastem technique with 2 enzymatic digestion protocols (Domenis et al., Stem Cell Research & Therapy, 2015).

The following year, the Lipogems technique is reported, which uses balls in a closed system to manually microfragment the adipose tissue. The publication from Tremolada's team (Tremolada et al., Curr Stem Cell Rep, 2016) already claims more than 7000 patients treated by this method all over the world, in plastic surgery, general surgery, orthopaedic surgery, but also maxillofacial surgery applications.

In 2016, Condé-Green's team has made a review about the mechanical extraction of cells from adipose tissue. The studies remain mainly in vitro and use at the same time centrifugation, stirring (manual or electrical) and vortex to isolate the cells (Condé-Green PRS 2016).

Finally, the emulsification technique described by Tonnard in 2013 (Tonnard et al., PRS, 2013) under the name of Nanofat, which consists in adipocyte lysis by inter-syringe shuffling, is also considered as a method for extracting cells from the stromal fraction, and in particular multipotent cells (Banyard et al., PRS, 2016). The Nanofat protocol has thereafter been marketed by Tulip Medical.

The literature of the field includes many articles, including:
Tonnard et al., PRS, 2013, Nanofat Grafting: Basic Research and Clinical Applications
Aronowitz et al., SpringerPlus, 2015, Mechanical versus enzymatic isolation of stromal vascular fraction cells from adipose tissue
Oberbauer et al., Cell Regeneration, 2015, Enzymatic and non-enzymatic isolation systems for adipose tissue-derived cells: current state of the art
Domenis et al., Stem Cell Research & Therapy, 2015, Adipose tissue derived stem cells: in vitro and in vivo analysis of a standard and three commercially available cell-assisted lipotransfer techniques
Cicione et al., PRS, 2016, In Vitro Validation of a Closed Device Enabling the Purification of the Fluid Portion of Liposuction Aspirates
Tremolada et al., Curr Stem Cell Rep, 2016, Adipose Tissue and Mesenchymal Stem Cells: State of the Art and Lipogems® Technology Development
Condé-Green et al., PRS, 2016, Shift toward Mechanical Isolation of Adipose-derived Stromal Vascular Fraction: Review of Upcoming Techniques
Banyard et al., PRS, 2016, Phenotypic Analysis of Stromal Vascular Fraction after Mechanical Shear Reveals Stress-Induced Progenitor Populations
Chaput et al., PRS, 2016, Mechanically isolated stromal vascular fraction provides a valid and useful collagenase-free alternative technique: A comparative study
Van Dongen et al., JTERM, 2017, Comparison of intraoperative procedures for isolation of clinical grade stromal vascular fraction for regenerative purposes: a systematic review.

### OBJECT OF THE INVENTION

In order to remedy the above-mentioned drawbacks of the state of the art and others that will appear upon reading of the present document, the present invention proposes a mixer with a particular structure operable to implement a mechanical method for preparing cells from sampled tissues, in particular by shearing of the tissues.

More particularly, it is proposed according to the invention a device for the mechanical fragmentation of tissues intended for the preparation of a composition of isolated cells, in particular adipocytes and/or cells contained in the adipose tissue, from a tissue sample, obtained in particular during a liposuction, said preparation including at least one step of mechanically fragmenting the tissues in the device, said device comprising a motorized support and a container, the motorized support comprising a motor having a rotor integral with a coupling member, the container internally including an axially rotatable rotary shaft and including radial arms capable of sweeping the inside of the container as the rotary shaft rotates, the motorized support and the container including complementary detachable fastening means for immobilizing the container on the motorized support, the coupling member and the rotary shaft being configured in such a way as to allow axial rotation of the rotary shaft as the motor rotor rotates.

According to the invention, the radial arms are of two types: vane-type arms and filamentary-type arms.

Other non-limitative and advantageous features of the device according to the invention, taken individually or according to all the technically possible combinations, are the following:
the container is metallic,
the container is made of plastic material,
the plastic material is polymeric,
the container is translucent,
the container is transparent,
the coupling member allows the detachable and rotational coupling of the rotary shaft to the motor rotor,
the container is impermeable to liquids,
the container includes two openings, the first opening being closed by a filter, the second opening being closed by a self-sealing septum, which can be pierced by a hollow needle or a trocar or a cannula and which seals automatically upon removal of the needle or the trocar or the cannula in such a way to be impermeable to gases, liquids, particles and cells,
the self-sealing septum is a silicone valve,
the second opening is moreover closed to the outside by a removable plug or cap,
as an alternative, the self-sealing septum is a self-closing valve or flap,
at least one portion of the rotary shaft is metallic,
at least one portion of the rotary shaft is made of plastic material,
the rotary shaft is metallic,
the rotary shaft is made of plastic material,
the rotary shaft is made of metal and plastic material,
the vane-type arms are made of plastic material or metal,
the filamentary-type arms are made of plastic material, in particular nylon or equivalent,
the filamentary-type arms are metallic,
the vane-type arms have a substantially straight radial extension,
the vane-type arms have a curved radial extension,
at least one of the vane-type arms includes at least one ridge extending in a plane substantially perpendicular to its radial extension,
the vane-type arms have a transverse cross-section chosen among the circular, oval, square, rectangular, polygonal, spindle-shaped, T-shaped cross-sections,
the vane-type arms have a longitudinal cross-section chosen among the straight, curved, helical cross-sections,
the filamentary-type arms have a transverse cross-section chosen among the circular, oval, square, rectangular, polygonal cross-sections,
the vane-type arms are rigid and the filamentary-type arms are flexible,
at least one vane-type arm segment is flexible,
at least one portion of the vane-type arm is articulated to the rotary shaft,
the vane-type arms and the filamentary-type arms are both rigid,
the rotative shaft is horizontal, the container is cylindrical or straight frustoconical in shape, with a first vertical end wall and a second vertical end wall separated by a cylindrical or frustoconical lateral wall and the rotary shaft is coaxial to the lateral wall,
the rotary shaft is inclined with respect to the horizontal but not vertical, the container is cylindrical or straight frustoconical in shape, with a first vertical end wall and a second, upper vertical end wall separated by a cylindrical or frustoconical lateral wall,
the rotary shaft is vertical, the container is cylindrical or straight frustoconical in shape, with a lower bottom wall, an upper wall and a cylindrical or frustoconical rising lateral wall, the rotary shaft being central and coaxial to the lateral wall, and the vane-type arms are arranged in the lower portion of the rotary shaft, whereas the filamentary-type arms are arranged above the vane-type arms,
the rotary shaft is single-piece,
the rotary shaft is consisted of several parts assembled together,
the support has a portion that includes the motor and the coupling member and that forms a motorized portion of the support,
when the container is fastened to the motorized support, the motorized portion of the support is arranged above the container, the rotary shaft being driven by the coupling member by the top of the container, the bottom wall of the container being continuous, the motorized support having in particular a general horizontal-U or Z shape so that the motorized portion covers the container,
when the container is fastened to the motorized support, the motorized portion of the support is arranged above the container, the rotary shaft being driven by the coupling member by the bottom of the container, the bottom wall of the container including an opening of the passage of the rotary shaft, said passage opening being sealed,
the coupling member provides a direct-contact mechanical coupling with the rotary shaft,
the coupling member provides a remote magnetic coupling with the rotary shaft, which avoids a seal for the rotary shaft due to the fact that the latter does not pass through the container wall,
the motor rotor is a part of the rotary shaft, the coupling being electromagnetic between a stator of the motor in the motorized support and the rotor on the rotary shaft of the container, which avoids a seal for the rotary shaft due to the fact that the latter does not pass through the container wall,
the lower bottom wall and the rising lateral wall are single-piece,
the upper wall forms a lid for the container,
the upper wall is added on the top of the rising lateral wall,
the upper wall is removable from the top of the rising lateral wall,
the container is frustoconical, with a 43-mm diameter on the bottom wall side and a 54-mm diameter on the upper wall side,
the useful volume height of the container is of 70.7 mm for a useful volume of 100 cc,
the overall height of the container is 97.5 mm,
each vane-type arm has at least one bevelled longitudinal edge, said at least one bevelled edge being on the lateral side of the arm first sweeping the inside of the container as the rotary shaft rotates,
the bevelled edge is a sharp edge,
the bevelled edge further includes indentations,
each filamentary-type arm is consisted of a nylon or metal wire,
the nylon or metal wires have a diameter comprised between 0.5 mm and 3 mm,
the nylon or metal wires are staggered along the rotary shaft height, the distance between two adjacent wire stages being constant along the height,
the nylon or metal wires are staggered along the rotary shaft height, the distance between two adjacent wire stages varying along the height,
the nylon or metal wires are staggered along the rotary shaft height, the distance between two adjacent wire stages being comprised between 5 mm and 20 mm,
the nylon or metal wires are vertically aligned along the rotary shaft height,
the nylon or metal wires are helically aligned along the rotary shaft height,
the cylindrical or frustoconical rising lateral wall of the container internally includes serrations,
the container is impermeable to liquids and the upper wall includes two openings, the first opening being closed by a filter, the second opening being closed by a self-sealing septum, which can be pierced by a piercing tubing and which seals automatically upon removal of the piercing tubing in such a way to be totally impermeable,
the piercing tubing is a hollow needle or a trocar or a cannula,
the self-sealing septum is made of elastomer,
the self-sealing septum is a silicone valve,
at least one of the two openings of the upper wall is externally covered with a tearable membrane seal,
at least one of the two openings of the upper wall is externally covered with a removable cap,
the upper wall of the container is a removable lid,
the container with its rotary shaft is single-use,
the container with its rotary shaft is sterilizable,
the motorized support includes a system for controlling the speed of rotation of the motor rotor,
the motorized support includes a system for measuring the speed of rotation of the motor rotor,
the motorized support includes a system for measuring the resisting torque against the rotation of the motor rotor,
the motorized support includes a system for detecting the presence of the container,
the system for controlling the speed of rotation of the motor rotor is configured to allow at least one of the following actions: rotation in a constant direction, rotation in alternate directions, rotation at constant speed, rotation at variable speed, rotation at at least one of several speeds according to a speed evolution program, wherein said program can include steps conditioned to results of measurements performed by a measurement system of the motorized support,
the motorized support includes a timer for adjusting the duration of rotation of the motor rotor,
the motorized support includes a heat regulation means for regulating the temperature of the container,
preferably, the motorized support includes pre-set rotation speed regulation system and rotation duration timer, wherein the device is automatic and the user cannot adjust the speed or the duration.

The invention also proposes a method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support and a container, the motorized support comprising a motor having a rotor integral with a coupling member, the container internally including an axially rotatable rotary shaft and including radial arms capable of sweeping the inside of the container as the rotary shaft rotates, the motorized support and the container including complementary detachable fastening means for immobilizing the container on the motorized support, the coupling member and the rotary shaft being configured in such a way as to allow axial rotation of the rotary shaft as the motor rotor rotates.

According to said method, a device according to any one of the preceding claims is implemented for the fragmentation of the tissues, and the sampled tissue is introduced into the container, the motor is powered on, the container having previously been fastened to the motorized support so that the coupling member can rotate the rotary shaft then, after the motor has been stopped, at least one portion of the container content is collected.

In variants, possibly combined together, of said method:
the rotation of the rotary shaft is always performed in the same direction,
the rotation of the rotary shaft is alternated in one direction then in the other,
the rotation of the rotary shaft is alternated over at least 360°,
the rotation of the rotary shaft is alternated over more than 360°.

### DETAILED DESCRIPTION OF AN EXEMPLARY EMBODIMENT

The following description with respect to the appended drawings, given by way of non-limitative examples, will permit a good understanding of what the invention consists in and of how it can be implemented.

In the appended drawings:

FIG. 1 shows an axial vertical cross-section, passing through the rotary shaft, of the container of the invention,

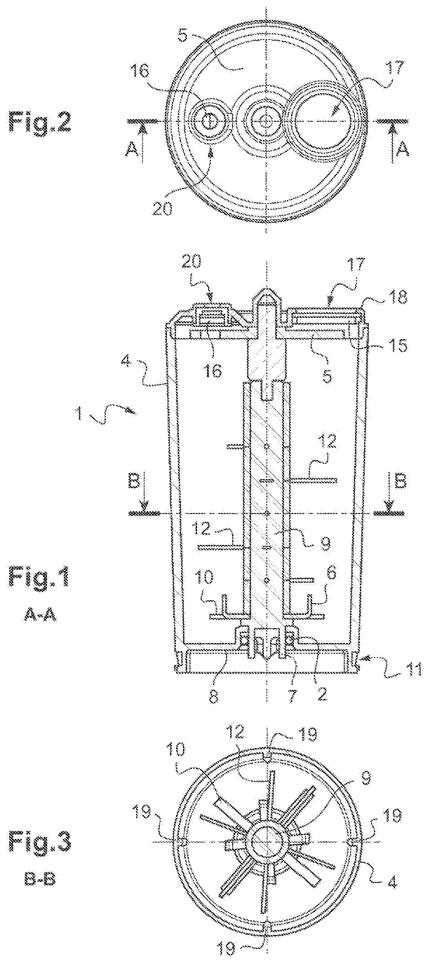

FIG. 2 shows a top view of the container of FIG. 1,

FIG. 3 shows a radial cross-section of the container of FIG. 1, and

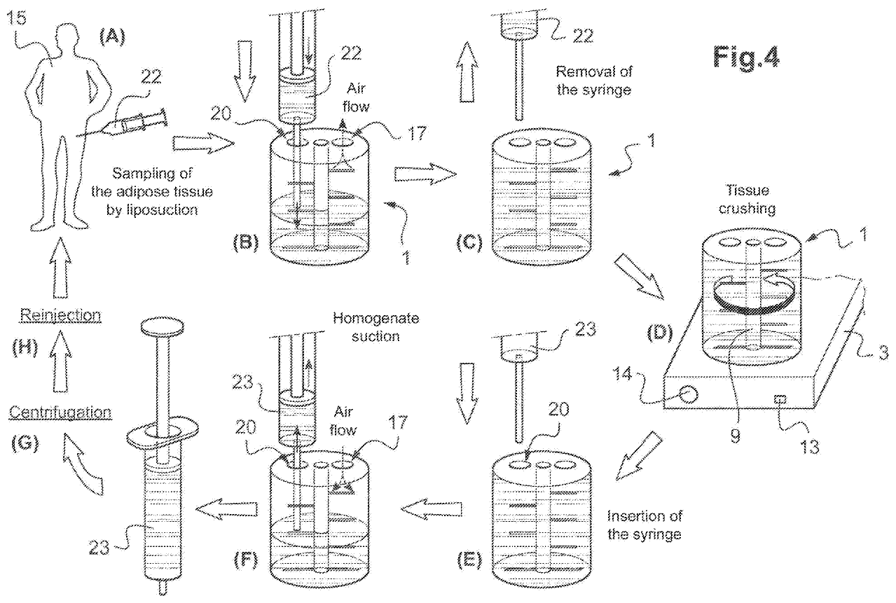

FIG. 4 schematizes the different steps of preparation of composition of isolated cells using the device of the invention.

### DEVICE

Within the framework of the solution of mechanical processing of the sampled tissue implemented in the invention, the separation of the cells from the tissue is based on attrition and shearing forces undergone by the tissue in a mixer.

Preferentially, this mixer is intended for crushing the adipose tissue and for obtaining cells composing the adipose tissue, mainly the cells of the SVF (Stromal Vascular Fraction) but it may also be used to purify cells contained in other tissues or organs.

The mixer of the invention mainly includes two components: a support including an electric motor, called motorized support, and a container capable adaptable on the motorized support. The motorized support includes a motor making it possible, when the container is fastened to the motorized support, to rotate a rotary shaft internal to the container, said rotary shaft including radial arms. The container is detachably fastened to the motorized support and a coupling means, which is disengageable from the rotational driving, is installed between the motorized support and the rotary shaft of the container.

The speed of rotation of the rotary shaft is preferentially of about 4000 rpm (rotation per minute), which provides an optimum yield of cell purification in terms of number of cells and of viability. In variants, a speed of rotation comprised between 2000 and 8000 rpm is provided, and the mixer may include means for adjusting the speed of rotation of the motor rotor and hence of the rotary shaft.

The rotary shaft, which is central/coaxial to the container, herein substantially frustoconical in shape, is provided with radial vanes at the base thereof, for a convection of the adipose tissue. Higher, along the rotative shaft, nylon or metal wires are fastened perpendicularly/radially to the rotary shaft in order to ensure efficient cutting and splitting of the tissue. The diameter of the nylon or metal wires is comprised between 0.5 mm and 3 mm. The number of nylon or metal wires is comprised between 3 and 15.

In order to optimize the cutting of the tissue by the nylon or metal wires, serrations may be inserted on the inner face of the container. The number thereof may be comprised between 2 and 12 and the thickness thereof may be comprised between 1 mm and 6 mm. The presence of the serrations makes it possible to create a turbulent flow of tissues allowing an optimal mixture. These serrations are nevertheless optional in the sense that it is possible to obtain cells without them, but their presence significantly increases the cell preparation yield.

The container has, in its upper part, a lid that includes two devices making it possible to reduce the contamination of the tissue during the implementation of the invention. The first device is a vent through the lid, provided with a filter of 0.22 μm, allowing exchanges of air devoid of infectious or inert particles. The second device is a valve or a septum allowing the passage of a cannula without passage of external air concomitantly with the passage of the cannula. Preferably, this valve or this septum let a 3-mm cannula, typically a liposuction cannula, pass through. This usable cannula diameter may however be from 1 mm to 5 mm. This valve or this septum allows the adipose tissue coming from the liposuction to enter directly the container for being crushed and makes it possible to sample the homogenate after mixing in the mixer.

Hence, preferably, the container is closed by a septum that is a silicone valve, accepting the passage of a hollow cannula, of diameter 1 to 5 mm, and that closes automatically upon removal of said cannula. This silicone valve may be completed by an external/top plug, to avoid a prolonged direct contact of the valve with the external air. This non-pierced silicone valve is impermeable, in particular in the outward direction from the container, to liquids, particles and cells. The silicone valve and the plug are impermeable to gas+liquid+particles+cells in the two directions.

In FIGS. 1 to 3 is shown an example of container 1 of the invention of vertical use and driving of its rotary shaft by the bottom. This container is herein consisted of a frustoconical single-piece bowl with a bottom wall 8 extended upward by a frustoconical rising lateral wall 4. The inner face of the lateral wall 4 of the container 1 includes serrations 19. The rotary shaft 9 is arranged centrally and coaxial to the lateral wall 4 and is herein formed by the assembly of several parts. The rotary shaft 9 passes through the bottom wall 8 in a sealed manner and includes, at its lower end, a means 7 for its fast coupling to a complementary coupling member (not shown) of a support 3 (see FIG. 4) including an electric motor and on which the container may be detachably fastened.

The motorized support 3 (see FIG. 4) includes means 13, 14 for the adjustment of its operating parameters, in particular on/off, duration and/or speed of rotation. To allow this detachable fastening, fastening means 11 that are complementary of those of the motorized support are implemented and, in the present case, with a quarter-turn screwing. The seal of the passage of the rotary shaft 9 through the bottom wall 8 is obtained by implementation of an O-ring seal 2.

The frustoconical bowl with a narrow lower base and a wide top is closed at its top by an upper wall 5 forming a lid. This lid 5 is in this example clipped and is not intended to be removed. It may even be welded. In a variant embodiment, this lid is removable. The lid 5 includes two through-openings. The first opening 17 is closed by a filter of 0.22 μm and a grating 18. The second opening 20 is closed by a self-closing valve 16 of 3-mm diameter and that ensures a total sealing when closed. This flap 16 gives way when a cannula is introduced into the second opening 20. In the case of a self-sealing septum of the silicone valve type, the latter closes after removal of the cannula. The second opening 20 is externally covered by a removable cap.

The rotary shaft 9 includes radial arms 10, 12 of two types. Towards the bottom of the rotary shaft are arranged rigid vane-type arms 10 and, above these latter, flexible or soft filamentary-type arm 12 made of nylon or metal. The vane-type arms 10 may include ridges 6 extending in a vertical plane substantially perpendicular to the radial extension of the arm 10. The radial arms, in particular the filamentary arms 12, are staggered over the height of the rotary shaft 9 and angularly distributed all over the latter.

### Method

The steps of obtaining the desired cell composition are schematized in FIG. 4.

Beforehand, at step A, a sampling of adipose tissue has been performed on a patient 21 by a suction syringe 22 or an automatic vacuum-sucking device. At step B, the adipose tissue is introduced into the container 1 by passage of the cannula of the syringe 22 through the second opening 20. During the injection of the tissue into the container, the air that is over-pressurized inside the container 1 exhausts through the first opening 17 by passing through the filter 15. At step C, the syringe 22 is removed from the container 1. At step D, the container 1 is fastened to the motorized support 3 and the motor of the latter is activated to rotate the rotary shaft 9 of the container 1, which destructs the tissue and releases the cells. The operating parameters are adjusted by the adjustment means 13, 14 of the motorized support. At step E, the cells and other elements of the tissue that has been broken up are collected into a syringe cannula 23 introduced through the second opening 20. At step F, during the sucking by means of the syringe 23, external air may enter the container through the first opening 17 by passing through the filter 15. At step G, a centrifugation is performed in order to select the desired cells for a reinjection at step H.

Based on a sampled adipose tissue, the mixer of the present invention allows obtaining a cellular composition called SVF (Stromal Vascular Fraction) close to that obtained by the protocol using collagenase.

Typically, from the quantitative point of view, based on 25 ml of adipose tissue, about 200 000 of viable cells are obtained. From the qualitative point of view, the use of the mixer of the invention makes it possible to obtain about 17% of cells having a phenotype CD34+, CD31−, CD45− with respect to the total number of purified cells gathering the cells considered as progenitor cells (Mesenchymal Stem Cells) as well as the pericytes. Such a ratio is very close of that obtained with the purification protocol based on the use of the collagenase (19%).

1. A device for the mechanical fragmentation of tissues intended for the preparation of a composition of isolated cells, in particular adipocytes and/or cells contained in the adipose tissue, from a tissue sample, obtained in particular during a liposuction, said preparation including at least one step of mechanically fragmenting the tissues in the device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container (1) as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein the radial arms (10, 12) are of two types: vane-type arms (10) and filamentary-type arms (12).

2. The device according to claim 1, wherein the vane-type arms (10) are rigid and the filamentary-type arms (12) are flexible.

3. The device according to claim 1, wherein the rotary shaft (9) is vertical, the container (1) is cylindrical or straight frustoconical in shape, with a lower bottom wall (8), an upper wall (5) and a cylindrical or frustoconical rising lateral wall (4), the rotary shaft (9) being central and coaxial to the lateral wall (4), and wherein the vane-type arms (10) are arranged in the lower portion of the rotary shaft (9), whereas the filamentary-type arms (12) are arranged above the vane-type arms (10).

4. The device according to claim 1, wherein each vane-type arm (10) has at least one bevelled longitudinal edge, said at least one bevelled edge being on the lateral side of the arm first sweeping the inside of the container as the rotary shaft (9) rotates.

5. The device according to claim 1, wherein each filamentary-type arm (12) is consisted of a nylon or metal wire.

6. The device according to claim 5, wherein the nylon or metal wires have a diameter comprised between 0.5 mm and 3 mm.

7. The device according to claim 3, wherein the cylindrical or frustoconical rising lateral wall (4) of the container (1) internally includes serrations.

8. The device according to claim 3, wherein the container (1) is impermeable to liquids and wherein the upper wall (5) includes two openings, the first opening (17) being closed by a filter (15), the second opening (20) being closed by a self-sealing septum which can be pierced by a piercing tubing and which seals automatically upon removal of the piercing tubing in such a way to be totally impermeable.

9. The device according to claim 8, wherein, as an alternative, the self-sealing septum is a self-closing valve (16) or flap.

10. The device according to claim 2, wherein each filamentary-type arm (12) is consisted of a nylon or metal wire.

11. The device according to claim 10, wherein the nylon or metal wires have a diameter comprised between 0.5 mm and 3 mm.

12. The device according to claim 2, wherein the rotary shaft (9) is vertical, the container (1) is cylindrical or straight frustoconical in shape, with a lower bottom wall (8), an upper wall (5) and a cylindrical or frustoconical rising lateral wall (4), the rotary shaft (9) being central and coaxial to the lateral wall (4), and wherein the vane-type arms (10) are arranged in the lower portion of the rotary shaft (9), whereas the filamentary-type arms (12) are arranged above the vane-type arms (10).

13. The device according to claim 12, wherein each filamentary-type arm (12) is consisted of a nylon or metal wire.

14. The device according to claim 13, wherein the nylon or metal wires have a diameter comprised between 0.5 mm and 3 mm.

15. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 1 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.

16. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 2 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.

17. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 3 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.

18. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 4 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.

19. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 5 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.

20. A method for preparing a composition of isolated cells, in particular adipocytes and/or cells contained in the adipose tissue, from a tissue sample, obtained in particular during a liposuction, said preparation including at least one step of mechanical fragmentation of the tissues in a device, said device comprising a motorized support (3) and a container (1), the motorized support (3) comprising a motor having a rotor integral with a coupling member, the container (1) internally including an axially rotatable rotary shaft (9) and including radial arms (10, 12) capable of sweeping the inside of the container as the rotary shaft (9) rotates, the motorized support (3) and the container (1) including complementary detachable fastening means (11) for immobilizing the container (1) on the motorized support (3), the coupling member and the rotary shaft (9) being configured in such a way as to allow axial rotation of the rotary shaft (9) as the motor rotor rotates,
wherein a device according to claim 6 is implemented for the fragmentation of the tissues, and the sampled tissue is introduced into the container (1), the motor is powered on, the container (1) having previously been fastened to the motorized support (3) so that the coupling member can rotate the rotary shaft (9) then, after the motor has been stopped, at least one portion of the container (1) content is collected.
